(19) United States
(12) Patent Application Publication
(10) Pub. No.: US 2017/0128325 A1
(43) Pub. Date: May 11, 2017

(54) REMINDER DEVICE

(71) Applicant: SANOFI-AVENTIS DEUTSCHLAND GMBH, Frankfurt am Main (DE)

(72) Inventors: Stephan Riedel, Frankfurt am Main (DE); Frank Oelgarth, Frankfurt am Main (DE)

(21) Appl. No.: 15/317,648

(22) PCT Filed: Jun. 12, 2015
(86) PCT No.: PCT/EP2015/063142

§ 371 Date: Dec. 9, 2016

(30) Foreign Application Priority Data
Jun. 17, 2014 (EP) 14172662.0

(51) Int. Cl. A61J 7/04; A61M 5/32; B65D 25/54; B65D 85/62; B65D 25/24; B65D 43/16; B65D 43/18; A61M 5/24; B65D 25/20

(52) U.S. Cl. CPC A61J 7/04; A61M 5/24; A61M 5/3204; B65D 25/54; B65D 25/205; B65D 25/24; B65D 43/16; B65D 43/18; B65D 85/62; A61M 2205/583

(57) ABSTRACT

The present invention relates to a reminder device to indicate to a user a point in time to administer a medicament by means of a drug delivery device (100), the reminder device comprises:—a container (12) extending in a longitudinal direction (1) having an inner cross-section (14) matching with a cross section (52) of a disposable component (50) of the drug delivery device (100), wherein the container (12) is adapted to accommodate at least two of the disposable components (50),—an indication (20) attached to the container (12), extending in longitudinal direction (1) and comprising at least two indicating items (22a, 22b, 22c, 22d) separated in longitudinal direction (1) by a distance (d) that corresponds to a geometric parameter (D) of the disposable component (50).

### FIELD OF THE INVENTION

The present invention relates to the field of reminder devices in order to remind a person or a patient to administer a medicament, typically by way of injection and by making use of a particular drug delivery device, e.g. by making use of an injection device. The invention further relates to a reminder system as well as to a method of using a reminder device.

### BACKGROUND

Injection devices for setting and dispensing a single or multiple doses of a liquid medicament are as such well-known in the art. Generally, such devices have substantially a similar purpose as that of an ordinary syringe.

Injection devices, in particular pen-type injectors have to meet a number of user-specific requirements. For instance, with patient's suffering chronic diseases, such like diabetes, the patient may be physically infirm and may also have impaired vision. Suitable injection devices especially intended for home medication therefore need to be robust in construction and should be easy to use. Furthermore, manipulation and general handling of such a device and its components should be intelligible and easy understandable. Moreover, a dose setting as well as a dose dispensing procedure must be easy to operate and has to be unambiguous.

Typically, such devices comprise a housing or a particular cartridge holder, adapted to receive a cartridge at least partially filled with the medicament to be dispensed. The device further comprises a drive mechanism, usually having a displaceable piston rod which is adapted to operably engage with a piston of the cartridge. By means of the drive mechanism and its piston rod, the piston of the cartridge is displaceable in a distal or dispensing direction and may therefore expel a predefined amount of the medicament via a piercing assembly, which is to be releaseably coupled with a distal end section of the housing of the injection device.

The medicament to be dispensed by the injection device is provided and contained in a cartridge. Such cartridges typically comprise a vitreous barrel sealed in distal direction by means of a pierceable seal and being further sealed in proximal direction by the piston. With reusable injection devices an empty cartridge is replaceable by a new one. In contrast to that, injection devices of disposable type are to be entirely discarded when the medicament in the cartridge has been dispensed or used-up.

The time schedule according to which particular medicaments have to be taken or have to be administered depends on the type of disease, the physiologic properties of the patient and the type of medicament. While some medicaments have to be taken several times a day according to a predefined time schedule, other medicaments need administering e.g. only once a day, every second day, once a week or even once a month. Especially with rather large time intervals between successive intakes of the medicament the risk that the patient simply forgets to take the medicament may become significant.

Typically, with drug delivery devices, such like injection devices, the device either has to be unlocked by removing a protective cap or the device has to be prepared with a disposable piercing assembly, which has to be detached from the device and which is to be discarded after an injection procedure took place.

Detachment and removal of protective caps for preparation of the device prior and/or after use thereof produces waste that has to be manually and thoroughly discarded by the end user or patient. If such disposable components comprise a tipped needle element further and separate handling of such disposable components bears a certain risk to health. Persons dealing with such disposable components may become subject to stitch damages.

From the general state of the art pill dispensers are known having a reminder device to be mounted on a container for pills and serving to provide a reminder of temporary record of the date or time medicine was last taken or next due. For example, U.S. Pat. No. 5,482,163 describes a last indicator apparatus which includes an indicator ring which is disposed over the outer surface of a cylindrical support of a pill dispenser and which is rotatable about the longitudinal axis over the outer surface to each of a plurality of selected positions. Such an indicator apparatus comprises at least one movable component at its outer circumference, that requires a particular handling and setting by the user or patient.

It is therefore an object of the present invention to provide a much simpler reminder device that is easily understandable and that provides unequivocal indication to a user or patient about a particular point in time, at which a next intake or administering of a particular medicament is due or at which the last intake or administering took place. The reminder device should be rather robust in construction. It should be rather easy in assembly, use and manufacture. Moreover, the reminder device should provide a well-defined handling of used disposable components of a drug delivery device.

### SUMMARY OF THE INVENTION

In a first aspect the invention relates to a reminder device to indicate to a user a point in time to administer a medicament by making use of a drug delivery device. The reminder device comprises a container extending in a longitudinal direction. The container has an inner cross section or at least an inner diameter that matches with a cross-section of a disposable component of the drug delivery device. Moreover, the container is adapted to accommodate at least two of the disposable components. Typically the container is adapted to accommodate and to receive a series of identically-shaped disposable components, that may have been used with one and the same drug delivery device or with a series of identical drug delivery devices.

The reminder device further comprises an indication attached to the container or embedded in the container. The indication extends in longitudinal direction, hence along the longitudinal extent of the container and comprises at least two indicating items separated in longitudinal direction by a distance (d) that corresponds to a geometric parameter (D) of the disposable component.

The outer geometry of the disposable component of the drug delivery device and the inner geometry of the container mutually match in such a way, that the disposable component is insertable into the container only in a predefined, typically in a single orientation. Hence, the outer cross-section or outer circumference of the disposable component directly matches with and corresponds with the inner cross-section of the container.

In this way, the disposable component is insertable into the container only in a well-defined or predefined orientation. For instance, the disposable component may be of cylindrical shape having an outer diameter that is only slightly smaller than the inner diameter of the cylindrically-shaped container. The container extending in longitudinal direction with its cylinder long axis may be open towards one end so as to allow for insertion of the disposable component into the container. Typically, with such a cylindrical embodiment the longitudinal or axial extension of the disposable component is larger than its diameter so that the disposable component is only insertable into the container in longitudinal direction when its cylinder long axis is arranged parallel to the cylinder long axis of e.g. the tubular-shaped container.

The relevant geometric parameter (D) of the disposable component may then be represented by its longitudinal extension, hence along the axial direction of its cylindrical geometry. The geometric parameter (D) may represent the axial or longitudinal height of the disposable component. When assembled inside the container, the disposable component is at least in sections visible or otherwise detectable from outside the reminder device. The position of the indication's indicating items typically matches with the position of the disposable component inside the container, so that for instance an edge or some other peculiar geometric feature of the disposable component geometrically coincides with the indicating item, thereby directly and unequivocally pointing to the particular indicating item, which typically represents a particular point in time.

Typically, the container is adapted to receive a stack of identically-shaped disposable components of the drug delivery device, in particular of an injection device. The distance between consecutive indicating items arranged in longitudinal direction then corresponds to the periodicity of the stack of multiple disposable components that can be stacked inside the container.

The number of disposable components stacked or arranged inside the container then always directly correlates and coincides with the position of a respective indicating item. The size of a stack of at least two disposable components inside the container is then always directly indicative of a particular indicating item that represents a particular point in time, typically at which the next intake of a medicament is due or at which the last intake took place.

The indication acts as a kind of fill level indicator of the container. The container is particularly adapted to collect used disposable device components. In this way, the reminder device with its container also serves as a waste bin, in which the disposable component can be safely stored. A danger of stitches or other injuries can be minimized in this way.

The distance between neighboring or adjacent indicating items of the indication either directly equals or corresponds to the respective extension of the disposable component in longitudinal direction when assembled and received in the container. In case of a nested and stacked configuration of several disposable components it is conceivable, that the distance (d) is smaller than the total extension of the disposable component. In this case the distance (d) substantially equals the periodicity of the stack of disposable components.

According to another embodiment the container comprises a transparent sidewall. In an alternative embodiment the sidewall, comprises at least one transparent portion extending in longitudinal direction. By either having a transparent sidewall or by having a transparent portion in the sidewall the arrangement of disposable components inside the container can be visually inspected from outside the reminder device. The transparent portion of the sidewall may either comprise a transparent window or a recess, e.g. a longitudinal slit, through which the interior of the container is visually inspectable.

Generally, the sidewall completely encloses or forms the interior of the container. The sidewall may comprise a circular or oval cross-section. Depending on the cross-section of the disposable component the interior of the sidewall may also comprise a particular geometric structure, such like a triangular structure, a rectangular structure, a pentagon structure, a hexagon structure or some arbitrary regular or irregular multiple-edge structure. Moreover, the cross-section of the inside facing portion of the sidewall may be symmetric or asymmetric so as to limit insertion of the disposable components only in a particular and predefined orientation.

According to another embodiment the indication extends along the outside of the container and comprises a persistent, writable or modifiable label. In embodiments where the sidewall is completely transparent, the indication may be arranged at an arbitrary position along the outer circumference of the sidewall of the container. The indication only extends in longitudinal direction so that respective indicating items thereof match and coincide with a particular geometric portion, e.g. with an edge of the disposable component when assembled inside the container. In other embodiments, wherein the container only comprises a longitudinally extending transparent portion it is of particular benefit when the indication is located circumferentially adjacent to the transparent portion. In this way, the position of a geometric characteristic of the disposable component, e.g. an edge of the disposable component with an indicating item is directly and unequivocally visible.

When the indicating items are located directly adjacent to the transparent portion of the container's sidewall they act like numbers of a scale.

The indication may be provided with a sticker or with a label with respective indicating items printed thereon. Moreover, it is conceivable, that the indication comprises blank indicating items at predefined longitudinal positions. Upon prescription by a healthcare professional the various indicating items may be manually and individually provided with information. In particular, a healthcare professional may write certain points in time next to blank indicating items. In this way, the reminder device can be individually prepared and adapted to a particular prescription schedule without the possibility of modification by third parties, by the patient or by the user.

The label may be persistent, hence unmodifiable. In other embodiments it is conceivable, that the label is erasable so that a reconfiguration of the indication is enabled. In typical application scenarios, the label, hence the indication and the indicating items are non-modifiable. Once the reminder device has been filled with disposable components, the reminder device together with the disposable components assembled and received therein is intended to be discarded.

In a respective waste bin to receive the reminder device the disposable components remain inside the reminder device in an ordered way, so that a damage to health, in particular stitch damages for persons dealing with respective garbage can be effectively reduced or can be completely eliminated.

According to another embodiment the container comprises a stand at a first longitudinal end. By way of the stand, the container can be assembled in an upright configuration. It can be positioned on a base, such like a table, thereby pointing with its longitudinal direction upwardly.

By means of such a stand, which may at the same time form a lower and axial end of the container and its sidewall the disposable components inserted into the container can be kept in an ordered way under the effect of gravity.

According to another embodiment the container comprises an insert opening at a second longitudinal end. First and second longitudinal ends are located on opposite end sections of the container, hence at opposite end sections of its sidewall. By having an insert opening at an upper longitudinal end of the container, disposable components can be inserted through the insert opening and into the container. Thereafter and under the effect of gravity they may easily approach the second longitudinal end, hence the bottom of the container. By having the insert opening near a top end of the reminder device, a well-defined stacking order of consecutively inserted disposable components can be attained. Insertion and storage of disposable components into the container can be supported and driven by gravity in a straight forward way. The inner cross section of the container and the outer cross section of the disposable component are mutually adapted such that the disposable component is unable to tilt or tumble inside the container when proceeding inside the container towards the bottom.

According to another embodiment the container further comprises a closure at the second longitudinal end. By means of the closure, which may be releasably or unreleasably attached to the second, hence the upper end of the container, unintentional removal of disposable components from the container can be effectively prevented. In the event, that a container with at least some disposable components therein is subject to a tilting over, the closure effectively prevents release of disposable components from inside the container.

The closure may comprise a lid, which is either pivotable with regard to a longitudinal or transverse axis with respect to the overall elongated geometry of the container. Alternatively, the closure may comprise a cup-shaped receptacle that is adapted to receive the outer circumference of the container's second longitudinal end.

The closure may either completely seal the upper and second longitudinal end of the container. In an alternative embodiment the closure comprises a through opening that exclusively allows to insert a disposable component but which effectively prevents removal of a disposable component from inside the container.

According to another embodiment the container is equipped with at least one safety catch at an inside-facing portion of the sidewall. The safety catch is operable to prevent release and withdrawal of the disposable component from the container. The safely catch may comprise one or several resiliently deformable latch or flap elements that support insertion of a disposable component into the container but which effectively block a removal thereof from the container.

The safety catch may comprise various barbs and may act in a manner comparable to barbed hooks.

The safety catch may be provided at an inside-facing portion of the closure, hence at or near the second longitudinal end of the container. The safety catch can be arranged along the circumference of an insert opening of the closure. Alternatively or in addition the safety catch is arranged at one or several inside-facing portions of the container sidewall. It is generally conceivable, that several safety catches are provided on the inside-facing portion of the container sidewall at a longitudinal distance. In particular, there may be provided a safety catch for every or for every second disposable component to be arranged inside the reminder device's container.

According to another embodiment the reminder device also comprises a detector to detect insertion of a disposable component into the container. In addition or alternatively, the detector may be adapted to determine a number of disposable components actually located and arranged inside the container. The detector may be operable to act as a counter, i.e. to detect every event when a disposable component is inserted into the container. In addition or alternatively, the detector may be adapted to sense the number of disposable components, e.g. by measuring their weight. The detector is either designed as a mechanical detector or as an electrical detector, operable to generate electrical signals in response to the insertion of a disposable component into the container. Electrical signals generated by the detector may be further processed by a control or by a transponder of the reminder device.

According to another embodiment the detector comprises at least one of a magnetic sensor, an induction coil or an inductor, a capacitive sensor, a photodiode or a piezo-electric element. Depending on the specific implementation of the detector, different ways to detect insertion of a disposable component into the container can be implemented. When implementing the detector as a magnetic sensor, e.g. comprising an induction coil it is of particular benefit, when the disposable component comprises some kind of magnetically detectable or inductive component, so that insertion of the disposable component into the container is detectable by the respective detector.

When implemented as a photodiode, it may be of particular benefit when the detector comprises a pair of a photodiode and a light source, e.g. arranged on geometrical opposite sides of the insert opening or beneath the insert opening. During insertion of the disposable component into the container, a signal obtainable from a photodiode significantly changes, thereby indicating the action of insertion.

When implemented as a piezo-electric element it is for instance conceivable to even measure the weight of a stack of disposable components inside the reminder device's container. A piezo-electric element may be for instance assembled near or at the bottom, hence in or at the stand of the container.

According to another embodiment the reminder device further comprises a control and a transponder. The transponder is operably connected to the control to communicate with an electronic device. The transponder is typically adapted and configured for wireless communication with an electronic device. It may be equipped with electromagnetic broadcasting and transmitting equipment operating under a standardized communication protocol, such like Bluetooth, wireless LAN, LTE, UMTS or other communication standards e.g. based on RF-based data transmission. It is also conceivable that the transponder is operable to communicate with the electronic device by a wired connection.

The control is particularly operable to process electrical signals obtained from one of the above mentioned detectors. Hence, the control is operably connected to at least one of the detectors in order to process respective detector or sensor signals.

It is also conceivable, that the detector is directly connected to the transponder so that the reminder device is substantially control-less or void of a control. Any electrical signals or data obtained and gathered by the detector can be transmitted to an electronic device, so that a data acquisition or data processing exclusively takes place in the remote electronic device. The electronic device may comprise a computer, a tablet computer, a mobile phone, a smartphone or some other data processing electronic device. In this way, customer electronic devices can be effectively used to process data obtained from one of the reminder device's detectors.

By transmitting any electrical signals or data obtained and generated by the detector to an electronic device not belonging to the reminder device, the overall design of the reminder device and any software implementation of electronic components of the reminder device can be kept on a low and cost efficient level. Moreover, through the communication with an electronic device, use of the reminder device can be communicated to the electronic device and further to a healthcare provider. In this way, compliance with a prescription schedule or application schedule can be surveyed and controlled even if the drug delivery device is exclusively used for home medication.

According to another embodiment the control is at least operable to transmit a command to the electronic device in response to the sensor detecting insertion of a disposable component into the container. In addition and by implementing a control, a transponder and a detector, various different modes of user feedback can be generated and provided. With the detector, the control and/or the transponder and optionally with the electronic device it is also conceivable to generate an audible or haptic indication to a user regarding a proceeding intake or administering of the medicament.

For instance, detection of insertion of the disposable component into the container may trigger playing of a song, of a jingle or may unlock for instance a certain level of a computer game running on the electronic device. In this way, the reminder device and its communication link with the electronic device provides a kind of reward for the user when inserting the disposable component into the container. Especially for children or teenagers, this functionality may increase the motivation to insert the disposable components into the container. In addition, the patient or user is highly encouraged to conduct a subsequent administering procedure when it is due.

According to another embodiment the reminder device further comprises an energy source or an energy storage. The reminder device may be equipped with a battery or with a solar panel, e.g. in order to charge an onboard electric battery. It is also conceivable, that the energy source is a rather passive source, such like it is known from RFID chip technology. The reminder device itself may be implemented as a passive RFID component, which is subject to a power up procedure when the electronic device, e.g. comprising a RFID reader, approaches the reminder device.

According to another aspect the invention also relates to a reminder system comprising at least one reminder device as described above. In addition to the reminder device the reminder system further comprises at least two disposable components releasably attachable to the drug delivery device. Alternatively the reminder system comprises at least two drug delivery devices, each of which being initially equipped with a detachable disposable component. The disposable component may comprise a needle assembly of an injection device, which is to be assembled to the device prior to use and which is to be disassembled and detached from the device after use.

After detachment it should be inserted into the container of the reminder device. The drug delivery device may be of disposable or reusable type. Especially with a sophisticated drug delivery device it is conceivable, that the reminder system comprises the reminder device and a set of detachable one-time-usable needle assemblies. Needle assemblies and the reminder device are then delivered as a kit to end users while the user himself makes constant use of one and the same drug delivery device.

In another embodiment the drug delivery device is already equipped with the detachable disposable component. Then after or prior to a single use or after or prior to consecutive uses of the drug delivery device, the disposable component has to be detached and to be inserted into the reminder device. The drug delivery device comprises an injection device for parenteral administering of a liquid medicament to biological tissue. The drug delivery device may comprise an auto injector, while the detachable disposable component comprises a needle shield remover cap, which prior to an initial or single use of the drug delivery device has to be detached therefrom and discarded.

In a further embodiment wherein the drug delivery device of the reminder device comprises a cartridge filled with the medicament. The cartridge may be pre-assembled in the drug delivery device, which itself may be of disposable or reusable type.

According to another independent aspect the invention further relates to a method of using a reminder device as described above. In a first step, the disposable component is detached from the drug delivery device after use of the drug delivery device. Thereafter and in a second step, the disposable component is inserted in a pre-defined orientation into the container of the reminder device. Said method is repeated each time the drug delivery device is used for administering of a liquid medicament. After consecutive uses, the disposable components stacked inside the interior of the reminder device's container visually indicate the next point in time at which a subsequent administering procedure should take place.

It has to be noted, that the above described reminder device and the reminder system directly correlates to the above mentioned method of using a reminder device. In this way, any benefits, features and effects mentioned in connection with the reminder device and the reminder system equally apply to the method of using the same; and vice versa.

The term “drug” or “medicament”, as used herein, means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-Y-glutamyl)-des(B30) human insulin; B29-N—(N-lithocholyl-Y-glutamyl)-des(B30) human insulin; B29-N-(w-carboxyheptadecanoyl)-des(B30) human insulin and B29-N-(w-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly- Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains p and E have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present invention without departing from the spirit and scope of the invention. Further, it is to be noted, that any reference numerals used in the appended claims are not to be construed as limiting the scope of the invention.

### BRIEF DESCRIPTION OF THE DRAWINGS

In the following, an exemplary embodiment of the present invention is described in detail by making reference to the Figs. in which:

FIG. 1 schematically illustrates a perspective view of a reminder device,

FIG. 2 shows another but rather schematic implementation of the reminder device, and

FIG. 3 schematically illustrates a drug delivery device with a detachable needle assembly as a disposable component.

### DETAILED DESCRIPTION

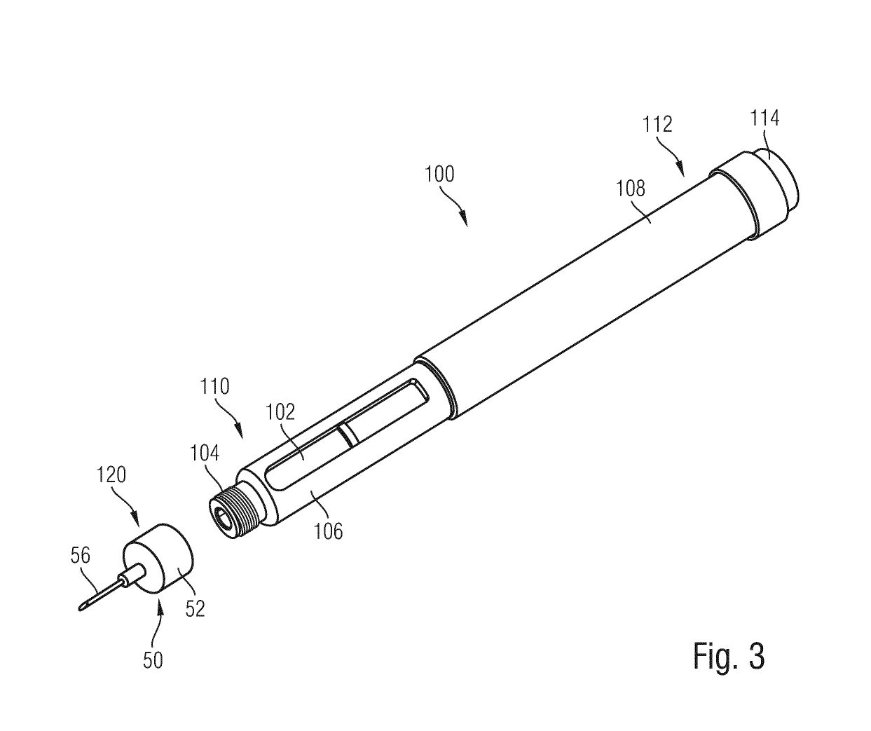

The drug delivery device 100 as schematically illustrated in FIG. 3 comprises a pen-shaped injector. The drug delivery device 100 comprises a distal housing portion, denoted as a cartridge holder 106 and which is adapted to house and to accommodate a vitreous cartridge 102. Inside the vitreous cartridge 102 there is located a liquid medicament to be dispensed via a detachable needle assembly 120. The needle assembly 120 is exemplary and illustrative of a disposable component 50 that can be attached and detached to a threaded socket 104 at a distal end 110 of the elongated and longitudinally extending drug delivery device 100. At a proximal end 112, the drug delivery device 100, in particular its proximal housing component 108 features a dose button 114, which is depressible in distal direction.

The dose button 114 located at the proximal end 112 of the drug delivery device 100 serves to trigger a drug delivery, hence an injection process. The needle assembly 120 further comprises an elongated and tipped needle 56, by way of which a pierceable septum located at a distal end of the cartridge 102 can be intersected and by way of which the medicament located in the cartridge 102 can be dispensed into biological tissue punctured by the distal end of the needle 56.

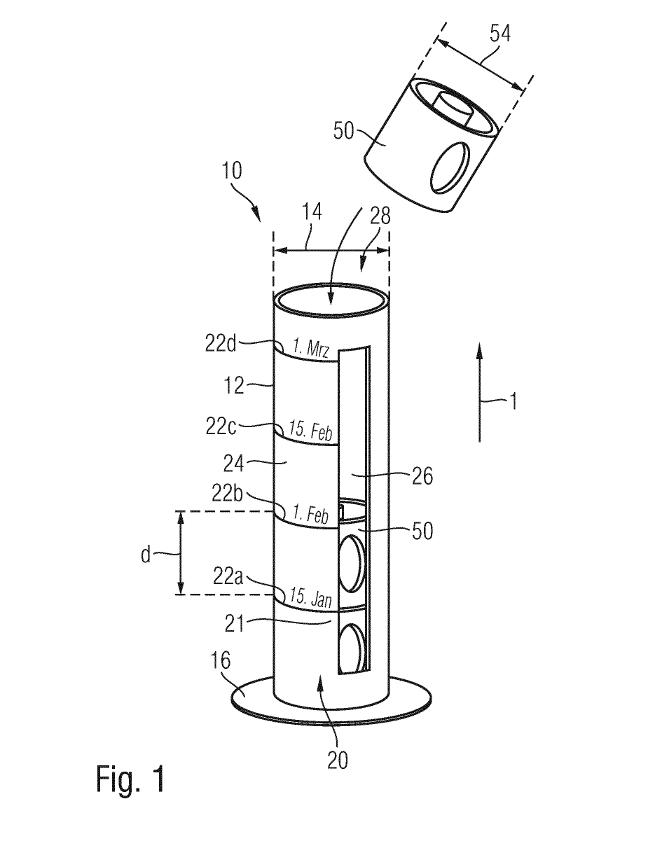

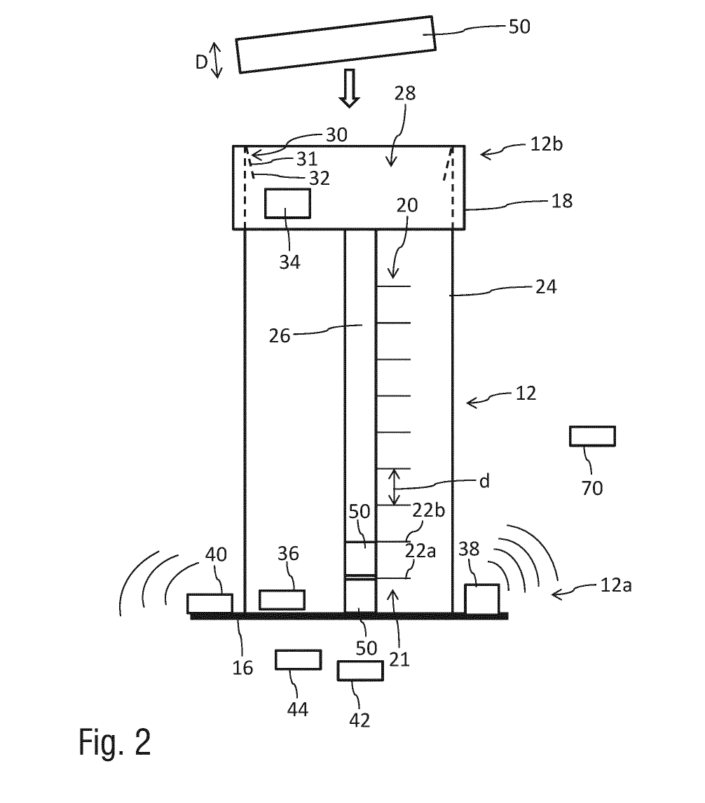

The disposable component 50, presently in form of a needle assembly 120 comprises a circular symmetric sleeve 52 having a particular cross-section 54 that fits into a container 12 of a reminder device as illustrated in FIGS. 1 and 2 only in well-defined orientation. The sleeve 52, which may be threadedly fastened to the threaded socket 104 of the drug delivery device 100 has to be oriented with its cylinder long axis parallel to the longitudinal direction 1 of the container 12.

The embodiment according to FIG. 3 is only illustrative. The reminder device 10 according to

FIGS. 1 and 2 is generally applicable to a large variety of different disposable components 50, such like shield remover caps of auto injectors. Such a needle shield remover cap as a disposable component 50 is schematically illustrated in FIG. 1. Prior to use of the drug delivery device or after use but before stowing away of the drug delivery device 100 the disposable component 50 is to be inserted into the interior of the container 12 of the reminder device 10. In the embodiment as shown in FIG. 1, the container 12 comprises a tubular shape with a unitary and surrounding sidewall 24 having a lower first longitudinal end 12a and an upper second longitudinal end 12b.

The container 12 extending in longitudinal direction (1) has an insert opening 28 near its second longitudinal end 12b. The inner cross-section 14 of the container is constant in longitudinal direction (1) and is slightly larger than the respective cross-section 54 of the disposable component 50. In this way, the disposable component 50 is insertable into the opening 28 and can be pushed further down along the interior of the container 12 in a well-defined orientation. According to the longitudinal extension D of the disposable components 50, the exterior of the container 12 comprises an indication 20 which is either attached to the outside of the container 12 or which is embedded or integrated into the container 12. The indication 20 comprises several indicating items 22a, 22b, 22c, 22d that are equidistantly spaced apart in longitudinal direction (1).

In the illustrated embodiment, the distance d between consecutive indicating items 22a, 22b, 22c, 22d equals the longitudinal size D of the disposable component 50. As several disposable components 50 are stacked inside the container 12, the ‘filling level’ and hence the number of the disposable components 50 inside the container 12 is directly apparent from outside the reminder device 10. The sidewall 24 comprises a longitudinally extending transparent portion 26, which is either formed by a transparent window or which is made by a longitudinally extending slit. As illustrated in FIGS. 1 and 2, the longitudinal position of consecutive indicating items 22a, 22b, 22c, 22d matches with upper and lower edges of consecutively arranged and stacked disposable components 50 inside the container 12.

As indicated in FIG. 1, each indicating item 22a, 22b, 22c, 22d is provided with a time or date indicia so as to indicate to a user, when a next administering of a particular medicament is due.

The indicating items can be provided on an adhesive strip or adhesive label 21 which is individually configurable, printable or writable and which can be attached along the longitudinal direction of the outer sidewall 24 of the container 12.

In the schematic illustration according to FIGS. 1 and 2, the container 12 is further equipped with a stand 16 standing in a plane substantially perpendicular to the longitudinal direction (1). In this way, the container 12 can be positioned in an upright orientation, e.g. on a base, such like a table top. The upright orientation is of particular benefit since disposable components 50 inserted via the upper insert opening 28 into the interior of the container 12 arrive at a stacked configuration as shown in FIGS. 1 and 2 under the effect of gravity.

Adjacent to the insert opening 28 the reminder device 10 as shown in FIG. 2 comprises a laterally inwardly and downwardly pointing safety catch 30. In the illustrated embodiment, the safety catch 30 either comprises a ring-shaped or several flap-like elements 31 that act as barbs or barb-like hooks. The downwardly pointing and inwardly pointing free ends 32 of the flap elements 31 are bendable from an initial configuration radially outwardly as the disposable component 50 is inserted from the top through the insert opening 28 of the closure 18 inside the container 12.

The outer dimension, hence the cross-section of the disposable component 50 and the inner circumference or cross-section 14 of the container mutually match in such a way, that it is impossible for the disposable component 50 to tumble and to re-orient inside the container 12. In this way, a rather smooth running insertion of the disposable component 50 into the container 12 can be provided. As the disposable component 50 is inserted into the container from the top as shown in FIG. 2, the flap elements 31 are bended temporarily radially outwardly so as to give way to the interior of the container 12.

Thereafter and due to an elastic restoring force, the flap elements 31 adapt their initial shape, in which their free ends 32 at least slightly protrude inward to the center of the container 12. In the event of a tilting over of the container, the free space between the free ends 32 of consecutively arranged flap elements 32 is smaller than the cross-section of the disposable component 50, thereby impeding a removal of the disposable component 50 from the interior of the container 12.

The closure 18 may be frictionally engaged with the outer circumference of the second longitudinal end 12b. The flap elements 31, hence the safety catch 30 may be either attached or integrally formed with the sidewall 24 of the container 12. Alternatively, the safety catch 30 could be attached to or integrally formed with the closure 18. The safety catch 30 may be arranged along the inner circumference of an insert opening 28 of either the closure 18 or of the second longitudinal end 12b of the container 12.

As it is further illustrated in FIG. 2, the reminder device 10 is equipped with various detectors 34, 36 as well as with a transponder 38 in order to communicate with an electronic device 70, e.g. in a wireless mode.

The detector 34, which is arranged near, at or in the closure 18 attached to the upper and second longitudinal end 12b of the container 12 is operable to detect insertion of the disposable component 50 into or through the insert opening 28 of the container 12, hence of the closure 18.

The detector 34 may comprise a magnetic sensor, an induction coil, a capacitive sensor, a photodiode or similar sensing arrangements operable to generate an electrical signal. The additional or alternatively implemented detector 36 arranged near a bottom or first longitudinal end 12a of the container 12 may comprise a piezo-electric element. This detector 36 may be either arranged at or in the sidewall 24 of the container 12. It may be attached to or integrated into the planar-shaped stand 16 forming a bottom of the interior of the container 12. By way of the detector 36 an overall weight of a stack of disposable components 50 can be detected and quantitatively measured.

Typically, the detectors 34, 36 are operably, hence electrically connected to a control 44, which is operable to process the electrical signals generated and provided by the detectors 34, 36. The reminder device 10 is further equipped with a transponder 38, which is connected to the control 44 or which is alternatively directly connected to at least one of the detectors 34, 36. By means of the transponder 38, a wireless communication link to an external electronic device 70, such like a mobile phone or computer can be established. Any data or sensor signals acquired from the detectors 34, 36 can either be directly communicated and transmitted to the electronic device 70. It is conceivable, that the reminder device 10 is void of an own onboard control 44. So at least one of the detectors 34, 36 may be directly interconnected with the transponder 38 in order to directly communicate with the electronic device 70. A control of the electronic device 70 may then execute data processing.

The reminder device 10 is further equipped with an indicator 40, by way of this additional indicator various different types of indications or reminders can be generated. The indicator 40 may comprise a visual indicator, and audible indicator or a haptic indicator. The indicator 40 may comprise a speaker so as to generate audible alerts to a user, that e.g. administering of the medicament is due. In addition, the indicator 40 may feature and provide a vibrational alert as well as a visual alert, such like a blinking light.

At least one of the control 44, indicator 40 and the electronic device 70 are operable to indicate to a user the preceding or next application or intake of the medicament.

As it is further illustrated in FIG. 2, the reminder device 10 may be equipped with an energy source 42. The energy source 42 may comprise a battery, a rechargeable battery and/or a solar panel. By means of the energy source 42, electric power for operation of at least one of the control 44 and one of the detectors 34, 36 or transponder 38 can be provided.

### LIST OF REFERENCE NUMBERS

1 longitudinal direction

10 reminder device

12 container

12a longitudinal end

12b longitudinal end

14 cross-section

16 stand

18 closure

20 indication

21 label

22a, b, c, d indicating item

24 sidewall

26 transparent portion

28 insert opening

30 safety catch

31 flap element

32 free end

34 detector

36 detector

38 transponder

40 indicator

42 energy source

44 control

50 disposable component

52 sleeve

54 cross-section

56 needle

70 electronic device

100 drug delivery device

102 cartridge

104 threaded socket

106 housing

110 distal end

112 proximal end

114 dose button

120 needle assembly

1. A reminder device to indicate to a user a point in time to administer a medicament by means of a drug delivery device (100), the reminder device comprises:
a container (12) extending in a longitudinal direction (1) having an inner cross-section (14) matching with a cross section (52) of a disposable component (50) of the drug delivery device (100), wherein the container (12) is adapted to accommodate at least two of the disposable components (50),
an indication (20) attached to the container (12), extending in longitudinal direction (1) and comprising at least two indicating items (22a, 22b, 22c, 22d) separated in longitudinal direction (1) by a distance (d) that corresponds to a geometric parameter (D) of the disposable component (50).

2. The reminder device according to claim 1, wherein the container (12) comprises a transparent side wall (24) or, wherein the side wall (24) of the container (12) comprises at least one transparent portion (26) extending in longitudinal direction (1).

3. The reminder device according to any one of the preceding claims, wherein the indication (20) extends along the outside of the container (12) and comprises a persistent, writable or modifiable label (21).

4. The reminder device according to any one of the preceding claims, wherein the container (12) comprises a stand (16) at a first longitudinal end (12a).

5. The reminder device according to any one of the preceding claims, wherein the container (12) comprises an insert opening (28) at a second longitudinal end (12b).

6. The reminder device according claim 5, wherein the container (12) comprises a closure (18) at the second longitudinal end (12b).

7. The reminder device according any one of the preceding claims, wherein the container (12) is equipped with at least one safety catch (30) at an inside facing portion of the side wall (24) to prevent release of the disposable component (50) from the container (12).

8. The reminder device according to any one of the preceding claims, further comprising a detector (34, 38) to detect insertion of a disposable component (50) into the container (12) and/or to determine a number of disposable components (50) located inside the container (12).

9. The reminder device according to claim 8, wherein the detector (34, 38) comprises at least one of a magnetic sensor, an induction coil, a capacitive sensor, a photodiode or a piezo-electric element.

10. The reminder device according to any one of the preceding claims, further comprising a control (44) and a transponder (38) operably connected to the control (44) to communicate with an electronic device (70).

11. The reminder device according to any one of the preceding claims, further comprising a energy source or energy storage (42).

12. The reminder device according to any one of the preceding claims, wherein the control (44) is operable to transmit a command to the electronic device in response to the sensor detecting insertion of a disposable component (50) into the container (12).

13. A reminder system comprising at least one reminder device according to any one of the preceding claims and further comprising:
at least two disposable components (50) releaseably attachable to the drug delivery device (10), or
at least two drug delivery devices (10) each of which being equipped with a detachable disposable component (50).

14. The reminder system according to claim 13, wherein the drug delivery device (100) comprises a cartridge (102) filled with the medicament.

15. Method of using a reminder device according to any one of the preceding claims 1 to 12 and comprising the steps of:
detaching of the disposable component (50) from the drug delivery device (10) after use of the drug delivery device (100), and
inserting the disposable component (50) in a pre-defined orientation into the container (12),
